(19) United States
(12) Patent Application Publication
(10) Pub. No.: US 2005/0112685 A1
(43) Pub. Date: May 26, 2005

(54) Compositions and methods for measuring analyte concentrations

(76) Inventors: Terry J. Amiss, Cary, NC (US); J. Bruce Pitner, Durham, NC (US); Tori C. Freitas, Raleigh, NC (US); Jennifer L. Giel, Madison, WI (US)

Correspondence Address: DAVID W. HIGHET;BECTON, DICKINSON AND COMPANY, 1 BECTON DRIVE, MC110, FRANKLIN LAKES, NJ, 07417, US

(21) Appl. No.: 10/721,091

(22) Filed: Nov. 26, 2003

(51) Int. Cl. G01N 33/53

(52) U.S. Cl. 435/7.1

(57) ABSTRACT

The current invention relates to fusion proteins comprising at least one functional periplasmic binding protein, at least one labeling moiety and at least one fluorescent protein. In one embodiment, the periplasmic binding protein is a functional glucose-galactose binding protein (GGBP). The invention also relates to methods for quantifying an analyte, for example glucose, in a cell or tissue comprising administering a composition comprising a fluorescent periplasmic binding fusion protein portion to the cell or tissue, and measuring the fluorescence of the fluorescent periplasmic binding fusion protein.

### BACKGROUND OF THE INVENTION

1. Field of the Invention

The current invention relates to fusion proteins comprising at least one functional periplasmic binding protein, at least one labeling moiety and at least one fluorescent protein. In one embodiment, the periplasmic binding protein is a functional glucose-galactose binding protein (GGBP). The invention also relates to methods for quantifying an analyte, for example, glucose, in a cell, tissue or biological fluid comprising administering a composition comprising a fluorescent periplasmic binding fusion protein portion to the cell or tissue, and measuring the fluorescence of the fluorescent periplasmic binding fusion protein.

### BACKGROUND OF THE INVENTION

Monitoring glucose concentrations to facilitate adequate metabolic control in diabetics is a desirable goal and would enhance the lives of many individuals. Currently, most diabetics use the “finger stick” method to monitor their blood glucose levels, and patient compliance is problematic due to pain caused by frequent (i.e., several times per day) sticks. As a consequence, there have been efforts to develop non-invasive or minimally invasive in vivo and more efficient in vitro methods for frequent and/or continuous monitoring of blood glucose or other glucose-containing biological fluids. Some of the most promising of these methods involve the use of a biosensor. Biosensors are devices capable of providing specific quantitative or semi-quantitative analytical information using a biological recognition element which is combined with a transducing (detecting) element.

The biological recognition element of a biosensor determines the specificity, so that only the compound measured leads to a signal. The selection may be based on biochemical recognition of the ligand where the chemical structure of the ligand (e.g., glucose) is unchanged, or biocatalysis in which the element catalyzes a biochemical reaction of the analyte.

The transducer then translates the recognition of the biological recognition element into a semi-quantitative or quantitative signal. Possible transducer technologies are optical, electrochemical, acoustical/mechanical or colorimetrical. The optical properties that have been exploited include absorbance, fluorescence/phosphorescence, bio/chemiluminescence, reflectance, light scattering and refractive index. Conventional reporter groups or labeling moieties such as fluorescent compounds may be used or, alternatively, there is the opportunity for direct optical detection, without the need for a label.

Biosensors specifically designed for glucose detection that use biological elements for signal transduction typically use electrochemical or colorimetric means of detecting glucose oxidase activity. This method is associated with difficulties including the influence of oxygen levels, inhibitors in the blood and problems with electrodes. In addition, detection results in consumption of the analyte that can cause difficulties when measuring low glucose concentrations.

A rapidly advancing area of biosensor development is the use of fluorescently labeled periplasmic binding proteins (PBPs). In order to accurately determine glucose concentration in biological solutions such as blood, interstitial fluids, ocular solutions or perspiration, etc., it is desirable to adjust the binding constant of the sensing molecule of a biosensor to match the physiological and/or pathological operating range of the biological solution of interest. Without the appropriate binding constant, a signal may be out of range for a particular physiological and/or pathological concentration. Additionally, biosensors may be configured using more than one protein, each with a different binding constant, to provide accurate measurements over a wide range of glucose concentrations. (See, e.g., U.S. Pat. No. 6,197,534 to Lakowicz).

Despite the usefulness of mutated PBPs, few of these proteins have been designed and examined, either with or without reporter groups. Specific mutations of sites and/or attachment of certain reporter groups may act to modify a binding constant in an unpredictable way. Additionally, a biosensor containing reporter groups may have a desirable binding constant, but not result in an easily detectable signal upon analyte binding. Some of the overriding factors that determine sensitivity of a particular reporter probe attached to a particular protein for the detection of a specific analyte is the nature of the specific interactions between the selected probe and amino acid residues of the protein. It is not currently possible to effectively predict how combining these interactions using existing computational methods affects protein function. It is also not possible to employ rational design methodology to optimize the choice of reporter probes. In fact, the effect of the reporter group on either the binding constant or the specificity of the binding protein is not predictable.

Therefore, there is a need in the art to design additional useful labeled mutated periplasmic binding proteins capable of generating a signal that can be accurately measured to determine the levels of analytes in a sample, including a patient's blood or interstitial fluid.

### SUMMARY OF THE INVENTION

The current invention relates to fusion proteins comprising at least one functional periplasmic binding protein, at least one labeling moiety and at least one fluorescent protein. In one embodiment, the periplasmic binding protein is a functional glucose-galactose binding protein (GGBP). The invention also relates to methods for quantifying an analyte, for example, glucose, in a cell, tissue or biological fluid comprising administering a composition comprising a fluorescent periplasmic binding fusion protein portion to the cell or tissue, and measuring the fluorescence of the fluorescent periplasmic binding fusion protein.

### BRIEF DESCRIPTION OF THE DRAWINGS

FIG. 1 depicts a representation of a fusion protein construct of DsRed2 and GGBP and a representation of the conformational change that GGBP undergoes when bound to glucose.

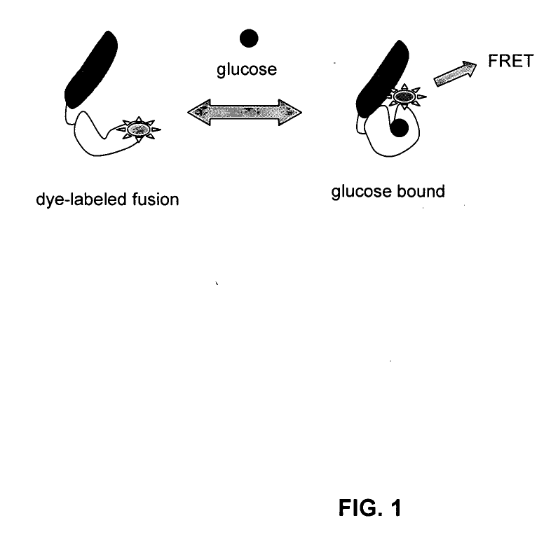

FIG. 2 depicts a DsRed2/GGBP tetramer.

FIG. 3 depicts a fluorescence emission spectrum of a fusion protein without a labeling moiety, a fusion protein with a labeling moiety and fusion protein with a labeling moiety bound to glucose.

FIG. 4 depicts the binding curves for DsRed2(C119A)GGBP(L238C)-acrylodan and DsRed2(C119A)GGBP(E149C,L238C)-acrylodan, and ligand glucose. A glucose affinity of 1 mM and 5.7 μM was demonstrated for each fusion protein, respectively.

FIG. 5 depicts the binding curve for DsRed2(C119A)GGBP(L238C)-acrylodan to glucose. This binding curve was graphed from the ratio of the acrylodan emission to DsRed2 emission. A glucose affinity of 4 μM was demonstrated for the fusion protein.

### DETAILED DESCRIPTION OF THE INVENTION

The current invention relates to methods for quantifying an analyte in a sample, with the methods comprising administering a fusion protein to the sample and measuring the level of fluorescence. The intensity of the measured luminescence is correlative to the amount of analyte in the sample. The fusion protein used in the methods of the current invention comprises a functional periplasmic binding protein (PBP), fused to at least one fluorescent protein, and at least one labeling moiety.

The detection of the analyte is made possible by the conformational change that the functional periplasmic binding protein undergoes when it binds to the analyte. This conformational change will, in turn, change the relative positions of the fluorescent protein and the labeling moiety, both attached to the functional PBP, to one another. This change in relative position permits an energy transfer from a donor molecule (the fluorescent protein or the labeling moiety) to the acceptor molecule (the labeling moiety or the fluorescent protein), which is then detectable via a signal, for example, fluorescence. The value of the fluorescence measured can be intensity or lifetime. Thus, the fluorescent measurement can be directly or indirectly tied to the concentration of measured analyte.

Furthermore, because the invention described herein utilizes two molecules capable of generating a signal, a change or transfer in energy may be detectable at two different wavelengths of the spectrum. For example, a change in energy emission at two or more different wavelengths can be compared, thereby creating a “ratiometric” measurement, which can be used to normalize values of measured analyte, as well as account for purity of the fusion protein or analyte.

As used herein, the quantification of an analyte can be a relative or absolute quantity. Of course, the quantity of analyte may be equal to zero, indicating the absence of the analyte sought. The quantity may simply be the measured fluorescent value, without any additional measurements or manipulations. Alternatively, the quantity may be expressed as a difference, percentage or ratio of the measured value of the analyte to a measured value of another compound including, but not limited to, a standard. The difference may be negative, indicating a decrease in the amount of measured analyte. The quantity may also be expressed as a difference or ratio of the analyte to itself, measured at a different point in time. The quantity of analyte may be determined directly from the measured fluorescent value, or the measured fluorescent value may be used in an algorithm, with the algorithm designed to correlate the measured fluorescent value to the quantity of analyte in the sample.

The analyte to be measured in the methods of the current invention include any compound capable of binding to the periplasmic binding protein portion of the fusion proteins used in the methods of the present invention. The binding of the analyte to the periplasmic binding protein portion may or may not be reversible. Examples of analytes include, but are not limited to, carbohydrates such as monosaccharides, disaccharides, oligosaccharides and polysaccharides, proteins, peptides and amino acids, including, but not limited to, oligopeptides, polypeptides and mature proteins, nucleic acids, oligonucleotides, polynucleotides, lipids, fatty acids, lipoproteins, proteoglycans, glycoproteins, organic compounds, inorganic compounds, ions, and synthetic and natural polymers. In one embodiment, the analyte is a carbohydrate. In particular, the carbohydrate analyte may be a sugar, such as glucose, galactose or ribose. More particularly, the analyte may be glucose.

The analyte is measured in a sample. As used herein, a sample can be any environment that may be suspected of containing the analyte to be measured. Thus, a sample includes, but is not limited to, a solution, a cell, a body fluid, a tissue or portion thereof, and an organ or portion thereof. When a sample includes a cell, the cell can be a prokaryotic or eukaryotic cell, for example, an animal cell. Examples of animal cells include, but are not limited to, insect, avian, and mammalian such as, for example, bovine, equine, porcine, canine, feline, human and nonhuman primates. The scope of the invention should not be limited by the cell type assayed. Examples of biological fluids to be assayed include, but are not limited to, blood, urine, saliva, synovial fluid, interstitial fluid, cerebrospinal fluid, lymphatic fluids, bile and amniotic fluid. The scope of the methods of the present invention should not be limited by the type of body fluid assayed. The terms “subject” and “patient” are used interchangeably herein and are used to mean an animal, particularly a mammal, more particularly a human or nonhuman primate.

The samples may or may not have been removed from their native environment. Thus, the portion of sample assayed need not be separated or removed from the rest of the sample or from a subject that may contain the sample. For example, the blood of a subject may be assayed for glucose without removing any of the blood from the patient. Of course, the sample may also be removed from its native environment. Furthermore, the sample may be processed prior to being assayed. For example, the sample may be diluted or concentrated; the sample may be purified and/or at least one compound, such as an internal standard, may be added to the sample. The sample may also be physically altered (e.g., centrifugation, affinity separation) or chemically altered (e.g., adding an acid, base or buffer, heating) prior to or in conjunction with the methods of the current invention. Processing also includes freezing and/or preserving the sample prior to assaying.

The methods of the current invention rely on the administration of a fusion protein to a sample. As used herein, “administration” is used to indicate any means that brings the sample into contact or close proximity with the fusion protein of the current invention. Thus, for example, the fusion protein can be administered to the sample by adding the fusion protein to the sample, or the fusion protein may be administered to the sample by placing the sample on or near the fusion protein. Furthermore, the fusion protein can be administered to the sample by using various structures or apparatuses that can more effectively place the fusion protein in an environment containing the sample. For example, in one embodiment of the current invention, the fusion protein is coated in or on an optical fiber, and the optical fiber can then be inserted into an environment that contains the sample, including, but not limited to, a subject's body. In another embodiment, the fusion protein of the present invention can be administered in an in vitro setting. Thus, the methods of the current invention can be utilized in an in vivo or an in vitro environment.

Furthermore, the analytes may be measured or monitored continuously using the methods of the current invention. For example, the fusion protein may be continuously bound to the analyte and, in turn, continuously emit a signal that may or may not be continuously detected, depending upon the detection device.

The fusion proteins of the current invention must possess: at least one functional periplasmic binding protein (PBP), at least one labeling moiety and at least one luminescent (e.g., fluorescent) protein. As used herein a functional PBP is a protein characterized by its three-dimensional configuration (tertiary structure), rather than its amino acid sequence (primary structure) and is characterized by a lobe-hinge-lobe region. The PBP will normally bind an analyte specifically in a cleft region between the lobes of the PBP. Furthermore, the binding of an analyte in the cleft region will then cause a conformational change to the PBP that makes detection of the analyte possible. Periplasmic binding proteins of the current invention include any protein that possesses the structural characteristics described herein; and analyzing the three-dimensional structure of a protein to determine the characteristic lobe-hinge-lobe structure of the PBPs is well within the capabilities of one of ordinary skill in the art. Examples of PBPs include, but are not limited to, glucose-galactose binding protein (GGBP), maltose binding protein (MBP), ribose binding protein (RBP), arabinose binding protein (ABP), dipeptide binding protein (DPBP), glutamate binding protein (GluBP), iron binding protein (FeBP), histidine binding protein (HBP), phosphate binding protein (PhosBP), glutamine binding protein, oligopeptide binding protein (OppA), or derivatives thereof, as well as other proteins that belong to the families of proteins known as periplasmic binding protein like I (PBP-like I) and periplasmic binding protein like II (PBP-like II). The PBP-like I and PBP-like II proteins have two similar lobe domains comprised of parallel β-sheets and adjacent α helices. The glucose-galactose binding protein (GGBP) belongs to the PBP-like I family of proteins, whereas the maltose binding protein (MBP) belongs to the PBP-like II family of proteins. The ribose binding protein (RBP) is also a member of the PBP family of proteins. Other non-limiting examples of periplasmic binding proteins are listed in Table I.

TABLE I
Genes Encoding Common Periplasmic Binding Proteins

| Gene name | Substrate | Species |
|---|---|---|
| alsB | Allose | E. coli |
| araF | Arabinose | E. coli |
| AraS | Arabinose/fructose/xylose | S. solfataricus |
| argT | Lysine/arginine/ornithine | Salmonella |
| | | typhimurium |
| artl | Arginine | E. coli |
| artJ | Arginine | E. coli |
| b1310 | Unknown | E. coli |
| | (putative, multiple sugar) | |
| b1487 | Unknown | E. coli |
| | (putative, oligopeptide | |
| | binding) | |
| b1516 | Unknown | E. coli |
| | (sugar binding protein | |
| | homolog) | |
| butE | vitamin B12 | E. coli |
| CAC1474 | Proline/glycine/betaine | Clostridium |
| | | acetobutylicum |
| cbt | Dicarboxylate | E. coli |
| | (Succinate, malate, fumarate) | |
| CbtA | Cellobiose | S. solfataricus |
| chvE | Sugar | A. tumefaciens |
| CysP | Thiosulfate | E. coli |
| dctP | C4-dicarboxylate | Rhodobacter |
| | | capsulatus |
| dppA | Dipeptide | E. coli |
| FbpA | Iron | Neisseria |
| | | gonorrhoeae |
| fecB | Fe(III)-dicitrate | E. coli |
| fepB | enterobactin-Fe | E. coli |
| fhuD | Ferrichydroxamate | E. coli |
| FliY | Cystine | E. coli |
| GlcS | glucose/galactose/mannose | S. solfataricus |
| glnH | Gluconate | E. coli |
| (protein: GLNBP) | | |
| gntX | Gluconate | E. coli |
| hemT | Haemin | Y. enterocolitica |
| HisJ | Histidine | E. coli |
| (protein: HBP) | | |
| hitA | Iron | Haemophilus |
| | | influenzae |
| livJ | Leucine/valine/isoleucine | E. coli |
| livK | Leucine | E. coli |
| (protein: L-BP | | |
| malE | maltodextrin/maltose | E. coli |
| (protein: MBP) | | |
| mglB | glucose/galactose | E. coli |
| (protein: GGBP) | | |
| modA | Molybdate | E. coli |
| MppA | L-alanyl-gamma-D-glutamyl- | E. coli |
| | meso-diaminopimelate | |
| nasF | nitrate/nitrite | Klebsiella oxytoca |
| nikA | Nickel | E. coli |
| opBC | Choline | B. Subtilis |
| OppA | Oligopeptide | Salmonella |
| | | typhimurium |
| PhnD | Alkylphosphonate | E. coli |
| PhoS (Psts) | Phosphate | E. coli |
| potD | putrescine/spermidine | E. coli |
| potF | Polyamines | E. coli |
| proX | Betaine | E. coli |
| rbsB | Ribose | E. coli |
| SapA | Peptides | S. typhimurium |
| sbp | Sulfate | Salmonella |
| | | typhimurium |
| TauA | Taurin | E. coli |
| TbpA | Thiamin | E. coli |
| tctC | Tricarboxylate | Salmonella |
| | | typhimurium |
| TreS | Trehalose | S. solfataricus |
| tTroA | Zinc | Treponema pallidum |
| UgpB | sn-glycerol-3-phosphate | E. coli |
| XylF | Xylose | E. coli |
| YaeC | Unknown | E. coli |
| | (putative) | |
| YbeJ(Gltl) | glutamate/aspartate | E. coli |
| | (putative, superfamily: lysine- | |
| | arginine-ornithine-binding | |
| | protein) | |
| YdcS(b1440) | Unknown | E. coli |
| | (putative, spermidine) | |
| YehZ | Unknown | E. coli |
| | (putative) | |
| YejA | Unknown | E. coli |
| | (putative, homology to | |
| | periplasmic oligopeptide- | |
| | binding protein - Helicobactr | |
| | pylori) | |
| YgiS (b3020) | Oligopeptides | E. coli |
| | (putative) | |
| YhbN | Unknown | E. coli |
| YhdW | Unknown (putative, amino | E. coli |
| | acids) | |
| YliB (b0830) | Unknown (putative, peptides) | E. coli |
| YphF | Unknown (putative sugars) | E. coli |
| Ytrf | Acetoin | B. subtilis |

In one embodiment of the present invention, the methods and compositions utilize more than one functional PBP. For example, two, three, four or more functional PBPs may be linked, cross-linked or genetically engineered as fusions (fused) to one another, or they may be linked, cross-linked or genetically engineered as fusions (fused) to another molecule that has multiple attachment sites. In one specific embodiment of the current invention, one, two, three or four functional GGBP proteins are fused (via a peptide bond) to a DsRed2 (fluorescent protein) tetramer, as described below. The DsRed2 tetramer has four N-termini with which the functional GGBPs, or other functional PBPs, may be fused using such ordinary recombinant DNA techniques or chemical synthesis techniques.

Functional PBPs of the current invention include, but are not limited to, wild-type PBPs, or fragments thereof, provided that the fragment retain at least a fraction of the binding specificity and/or affinity of the wild-type PBP. Additional examples of functional PBPs include derivatives (mutants) of the wild-type PBP, provided that the derivative PBPs retain at least a fraction of the binding specificity and/or affinity of the wild-type PBP. The terms “derivative,” “mutant” and “variant” are used interchangeably herein.

As used herein, the terms “protein” and “polypeptide” are used interchangeably and are used to refer to any peptide or protein comprising two or more amino acids joined to each other by peptide bonds or modified peptide bonds. “Polypeptide” is also used to mean shorter chains, commonly referred to as peptides, oligopeptides or oligomers. As stated earlier, functional periplasmic binding proteins of the present invention include derivative PBPs that may comprise an amino acid sequence other than the naturally occurring amino acid sequence, provided that the addition, deletion or mutation of the wild-type amino acid sequences does not completely ablate the function of the periplasmic binding protein. In other words, the present invention also contemplates functional derivatives of periplasmic binding proteins, such that these derivatives still possess at least some specific affinity for the same analytes as the wild-type proteins. Thus, as used herein, functional periplasmic binding proteins include wild-type and functional derivatives thereof. Functional derivatives of the present invention may be made or prepared by techniques well known to those of skill in the art. Examples of such techniques include, but are not limited to, mutagenesis and direct synthesis.

The functional periplasmic binding proteins, or functional derivates thereof, may also be modified, either by natural processes, such as post-translational processing, or by chemical modification techniques which are well known in the art. Such modifications are well described in basic texts and in more detailed monographs, as well as in voluminous research literature. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also, a given polypeptide may contain more than one modification. Examples of modifications include, but are not limited to, glycosylation, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Polypeptides may even be branched as a result of ubiquitination, and they may be cyclic, with or without branching. See, e.g., T. E. Creighton, Proteins-Structure And Molecular Properties, 2nd Ed., W. H. Freeman and Company, New York (1993); Wold, F., “Posttranslational Protein Modifications: Perspectives and Prospects”, in Posttranslational Covalent Modification Of Proteins, B. C. Johnson, Ed., Academic Press, New York (1983); Seifter et al., Methods in Enzymol, 182:626-646 (1990) and Rattan et al., Ann NY Acad Sci., 663:48-62 (1992).

In one embodiment of the current invention, the functional mutant PBPs are derivatives of the GGBP protein. Exemplary mutations of the GGBP protein include a cysteine substituted for a lysine at position 11 (K11C), a cysteine substituted for aspartic acid at position 14 (D14C), a cysteine substituted for methionine at position 16 (M16C), a cysteine substituted for valine at position 19 (V19C), a cysteine substituted for asparagine at position 43 (N43C), a cysteine substituted for a glycine at position 74 (G74C), a cysteine substituted for a tyrosine at position 107 (Y107C), a cysteine substituted for threonine at position 110 (T110C), a cysteine substituted for serine at position 112 (S112C), a double mutant including a cysteine substituted for a serine at position 112 and serine substituted for an leucine at position 238(S112C/L238S), a cysteine substituted for a lysine at position 113 (K113C), a cysteine substituted for a lysine at position 137 (K137C), a cysteine substituted for glutamic acid at position 149 (E149C), a double mutant including a cysteine substituted for an glutamic acid at position 149 and a serine substituted for leucine at position 238 (E149C/L238S), a double mutant including a cysteine substituted for an glutamic acid at position 149 and a cysteine substituted for leucine at position 238 (E149C/L238C), a double mutant including a cysteine substituted for an glutamic acid at position 149 and a arginine substituted for alanine at position 213 (E149C/A213R), comprising a cysteine substituted for histidine at position 152 and a cysteine substituted for methionine at position 182 (H152C/M182C), a double mutant including a serine substituted for an alanine at position 213 and a cysteine substituted for a histidine at position 152 (H152C/A213S), a cysteine substituted for an methionine at position 182 (M182C), a cysteine substituted for an alanine at position 213 (A213C), a double mutant including a cysteine substituted for an alanine at position 213 and a cysteine substituted for an leucine at position 238 (A213C/L238C), a cysteine substituted for an methionine at position 216 (M216C), a cysteine substituted for aspartic acid at position 236 (D236C), a cysteine substituted for an leucine at position 238 (L238C) a cysteine substituted for a aspartic acid at position 287 (D287C), a cysteine substituted for an arginine at position 292 (R292C), a cysteine substituted for a valine at position 296 (V296C), a triple mutant including a cysteine substituted for an glutamic acid at position 149 and an arginine substituted for an alanine at position 213 and a serine substituted for leucine at position 238 (E149C/A213R/L238S). These derivative GGBPs are described in U.S. patent application Publication Nos. 2003/0153026, 2003/0134346 and 2003/0130167, which are hereby incorporated by reference. When GGBP or a functional derivative thereof is to be the functional PBP used in the current invention, the analyte to be detected is either glucose or galactose.

One of the purposes of using derivative polypeptides in the methods and compositions of the current invention is to incorporate a labeling moiety onto or within the fusion protein, such that the fusion protein is labeled with a labeling moiety. With the addition/substitution of one or more cysteine residues into the primary structure of the functional periplasmic binding protein, some of the labeling moieties used in the current methods and compositions can be attached through chemical means, such as reduction, oxidation, conjugation, and condensation reactions. For example, any thiol-reactive group can be used to attach labeling moieties, e.g., a fluorophore, to a naturally occurring or engineered cysteine in the primary structure of the polypeptide.

The fusion proteins of the current invention also comprise a labeling moiety. A labeling moiety, as used herein, is intended to mean a chemical compound or ion that possesses or comes to possess a detectable non-radioactive signal. Examples of labeling moieties include, but are not limited to, transition metals, lanthanide ions and other chemical compounds. The non-radioactive signal includes, but is not limited to, fluorescence, phosphorescence, bioluminescence and chemiluminescence. In one embodiment, the labeling moiety is a fluorophore selected from the group consisting of fluorescein, coumarins, rhodamines, 5-TMRIA (tetramethylrhodamine-5-iodoacetamide), Quantum Red™, Texas Red™, Cy3, N-((2-iodoacetoxy)ethyl)-N-methyl)am-ino-7-nitrobenzoxadiazole (IANBD), 6-acryloyl-2-dimethylaminonaphthalene(acrylodan), pyrene, Lucifer Yellow, Cy5, Dapoxyle (2-bromoacetamidoethyl)sulfonamide, (N-(4,4-difluoro-1,3,5,7-tetramethyl-4-bora-3a,4a-diaza-s-indacene-2-yl)iodoacetamide (Bodipy507/545 IA), N-(4,4-difluoro-5,7-diphenyl-4-bora-3a,4a-diaza-s-indacene-3-propionyl)-N-′-iodoacetylethylenediamine (BODIPY® 530/550 IA), 5-((((2-iodoacetyl)amino)ethyl)amino)naphthalene-1-sulfonic acid (1,5-IAEDANS), and carboxy-X-rhodamine, 5/6-iodoacetamide (XRIA 5,6). Other luminescent labeling moieties include lanthanides such as europium (Eu3+) and terbium (Tb3+), as well as metal-ligand complexes of ruthenium [Ru(II)], rhenium [Re(I)], or osmium [Os(II)], typically in complexes with diimine ligands such as phenanthroline.

In particular, the fluorophore labeling moiety can be fluorescein, acryoldan, rhodamine, BODIPY, eosin, pyrene, acridine orange, PyMPO, alexa fluor 488, alexa fluor 532, alexa fluor 546, alexa fluor 568, alexa fluor 594, alexa fluor 555, alexa fluor 633, alexa fluor 647, alexa fluor 660, or alexa fluor 680. More particularly, the labeling moiety may be acrylodan. In another embodiment, the labeling moiety is an electrochemical moiety such that a change in the environment of this labeling moiety will change the redox state of the moiety.

In one embodiment of the current invention, the measurable signal of the fusion protein is actually a transfer of excitation energy (resonance energy transfer) from a donor molecule to an acceptor molecule. In particular, the resonance energy transfer is in the form of fluorescence resonance energy transfer (FRET). When FRET is used to quantify the analyte in the methods of the current invention, the labeling moiety can be the donor or the acceptor. The terms “donor” and “acceptor,” when used in relation to FRET, are readily understood in the art. Specifically, a donor is the molecule that will absorb a photon of light and subsequently initiate energy transfer to the acceptor molecule. The acceptor molecule is the molecule that receives the energy transfer initiated by the donor and, in turn, emits a photon of light. The efficiency of FRET is dependent upon the distance between the two fluorescent partners and can be expressed mathematically by: E=R₀⁶/(R₀⁶+r⁶), where E is the efficiency of energy transfer, r is the distance (in Angstroms) between the fluorescent donor/acceptor pair and R₀ is the Förster distance (in Angstroms). The Förster distance, which can be determined experimentally by readily available techniques in the art, is the distance at which FRET is half of the maximum possible FRET value for a given donor/acceptor pair.

The fusion proteins of the current invention also include at least one fluorescent protein. The fusion proteins may include two, three, four or more fluorescent proteins. If the fusion proteins of the current invention contain more than one fluorescent protein, the fluorescent proteins may or may not be chemically identical. Fluorescent proteins are easily recognized in the art. Examples of fluorescent proteins that are part of fusion proteins of the current invention include, but are not limited to, green fluorescent proteins (GFP, AcGFP, ZsGreen), red-shifted GFP (rs-GFP), red fluorescent proteins (RFP, including DsRed2, HcRed1, dsRed-Express), yellow fluorescent proteins (YFP, Zsyellow), cyan fluorescent proteins (CFP, AmCyan), and a blue fluorescent protein (BFP), as well as the enhanced versions and mutations of these proteins. For some fluorescent proteins enhancement indicates optimization of emission by increasing the proteins' brightness or by creating proteins that have faster chromophore maturation. These enhancements can be achieved through engineering mutations into the fluorescent proteins.

Mutating the fluorescent protein can prevent it from being labeled with the labeling moiety. Indeed, the labeling moiety can be conjugated to a cysteine residue, including any cysteine residue within the DsRed2 fluorescent protein or the PBP; however, the presence of a labeling moiety in the DsRed2 protein may interfere with the detection of glucose. Creation of a DsRed2(C119A) mutant (i.e., mutating the cysteine at position 119 to alanine) can circumvent this potential interference by allowing the PBP portion of the fusion protein, e.g., GGBP, to be site-specifically conjugated with the labeling moiety, rather than the fluorescent protein portion. In one embodiment, the fluorescent protein used in the fusion proteins of the current invention is RFP, particularly, discosoma red fluorescent protein (DsRed2). In one particular embodiment, the DsRed2 fluorescent protein used in the methods and compositions of the present invention is the mutant DsRed2(C119A), where “(C119A)” indicates a cysteine to alanine mutation at amino acid position 119 mutation in the DsRed2 wild-type amino acid sequence. The DsRed2 protein, or mutations thereof, can exist as a tetramer, thus, in one embodiment, the fusion protein comprises four fluorescent proteins, such as DsRed2, or mutations thereof, for example, DsRed2(C119A). Similar to the labeling moieties of the current invention, the fluorescent proteins of the fusion protein, when used in FRET systems, may be either the donor or acceptor molecule. Thus, the methods and compositions of the current invention provide versatile systems that utilize FRET, such that the fluorescent energy transfer may be from the labeling moiety to the fluorescent protein, or from the protein to the labeling moiety.

The signal can be detected or measured using any means that detects the energy transfer, such as a fluorometer, which can detect fluorescent intensity. The signal may also be measured or detected visually, without the aid of equipment.

In one embodiment of the current invention, device in which the functional GGBP(s) may be immobilized is a sensor attached to a collection of optical fibers. The fiber used in this embodiment may be a bifurcated fiber optic bundle. In one particular embodiment, the fiber optic contains six outer fibers arranged around a central fiber. The six fibers can be used as the excitation conduit and the central fiber as the detection conduit. These collection optics may also include additional fibers and/or lenses. The fiber can be polished, and then medical grade glue, or any other suitable adhesive, for example, Loctite 4011, can be applied to adhere the sensing element to one end of the fiber optic. The other end of the fiber bundle is connected to a fiber optic spectrophotometer. An LED at the appropriate wavelength (e.g., LS-450) can then be used and a fluorescence spectrometer can be used as a detector. Excitation sources may consist of, but are not limited to, for example arc lamps, laser diodes, or LEDs. Detectors may consist of, but are not limited to, for example, photodiodes, CCD chips, or photomultiplier tubes. A computer program, such as Ocean Optic OOIBase 32, may also be employed to trace the fluorescent emission.

The current invention also relates to compositions comprising a fusion protein portion and at least one labeling moiety. The fusion protein portions of the compositions of the current invention have been described herein.

The invention also relates to isolated nucleic acids coding for these fusion protein portions of the compositions previously described herein.

As used herein, “isolated nucleic acid molecule(s)” is used to mean a nucleic acid molecule, DNA or RNA, which has been removed from its native environment. For example, recombinant DNA molecules contained in a vector are considered isolated for the purposes of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. Isolated nucleic acid molecules according to the present invention further include such molecules produced synthetically.

“Nucleotide sequence” of a nucleic acid molecule or polynucleotide is used to mean a DNA molecule or polynucleotide, a sequence of deoxyribonucleotides and, for an RNA molecule or polynucleotide, the corresponding sequence of ribonucleotides (A, G, C and U), where each thymidine deoxyribonucleotide (T) in the specified deoxyribonucleotide sequence is replaced by the ribonucleotide uridine (U).

Nucleic acid molecules of the present invention may be in the form of RNA, such as mRNA, or in the form of DNA, including, for instance, cDNA and genomic DNA obtained by cloning or produced synthetically. The DNA may be double-stranded or single-stranded. Single-stranded DNA or RNA may be the coding strand, also known as the sense strand, or it may be the non-coding strand, also referred to as the anti-sense strand.

The present invention is further directed to fragments of the isolated nucleic acid molecules described herein. A “fragment” of an isolated nucleic acid molecule having the nucleotide sequence coding for the fusion proteins of the current invention is used to indicate fragments at least about 15 nucleotides (nt), and more preferably at least about 20 nt, still more preferably at least about 30 nt, and even more preferably, at least about 40 nt in length, which are useful as diagnostic probes and primers as discussed herein. Of course larger DNA fragments that are 50, 100, 150, 200, 250, 300, 350, 400, or 425 nt in length are also useful according to the present invention, as are fragments corresponding to most, if not all, of the nucleotide sequence that codes for a fusion protein of the current invention. A fragment at least 20 nt in length, for example, is understood to mean a fragment that include 20 or more contiguous bases from the nucleotide sequence coding for the fusion proteins of the current invention. Generating such DNA fragments would be routine to the skilled artisan. For example, restriction endonuclease cleavage or shearing by sonication could easily be used to generate fragments of various sizes. Alternatively, such fragments could be generated synthetically.

In another aspect, the invention provides an isolated nucleic acid molecule comprising a polynucleotide which hybridizes under stringent hybridization conditions to a portion of the polynucleotide in a nucleic acid molecule of the invention described above. “Stringent hybridization conditions” is understood in the art and is used to mean overnight incubation at 42° C. in a solution comprising: 50% formamide, 5×SSC (150 mM NaCl, 15 mM trisodium citrate), 50 mM sodium phosphate (pH 7.6), 5×Denhardt's solution, 10% dextran sulfate, and 20 g/ml denatured, sheared salmon sperm DNA, followed by washing the filters in 0.1×SSC at about 65° C.

A polynucleotide which hybridizes to a “portion” of a polynucleotide is understood to mean a polynucleotide (either DNA or RNA) hybridizing to at least about 15 nt, and more preferably at least about 20 nt, still more preferably at least about 30 nt, and even more preferably about 30-70 nt of the reference polynucleotide. Such fragments that hybridize to a portion of the reference polynucleotide are useful as fragments.

Of course, polynucleotides hybridizing to a larger portion of the reference polynucleotide, e.g., a portion 50-300 nt in length, or even to the entire length of the reference polynucleotide, are also useful as probes according to the present invention, as are polynucleotides corresponding to most, if not all, of the reference nucleotide sequences. A portion of a polynucleotide of “at least 20 nt in length,” for example, is understood to mean 20 or more contiguous nucleotides from the nucleotide sequence of the reference polynucleotide. As indicated, such portions are useful diagnostically either as a probe according to conventional DNA hybridization techniques or as primers for amplification of a target sequence by the polymerase chain reaction (PCR), as described, for instance, in Molecular Cloning, A Laboratory Manual, 3rd. edition, Sambrook, J., Fritsch, E. F. and Maniatis, T., eds., Cold Spring Harbor Laboratory Press, Cold Spring Harbor, N.Y. (2001), the entire disclosure of which is hereby incorporated herein by reference.

The present invention further relates to variants of the nucleic acid molecules of the present invention, which encode portions, analogs or derivatives of the fusion proteins. Variants may occur naturally, such as a natural allelic variant. An “allelic variant” is understood to mean one of several alternate forms of a gene occupying a given locus on a chromosome of an organism. See, e.g., Genes II, Lewin, B., ed., John Wiley & Sons, New York (1985). Non-naturally occurring variants may be produced using art-known mutagenesis techniques.

Such variants include those produced by nucleotide substitutions, deletions or additions. The substitutions, deletions or additions may involve one or more nucleotides. The variants may be altered in coding regions, non-coding regions, or both. Alterations in the coding regions may produce conservative or non-conservative amino acid substitutions, deletions or additions.

Thus, the invention contemplates isolated nucleic acid molecules comprising a polynucleotide having a nucleotide sequence at least about 95% identical, and more particularly, at least about 96%, about 97%, about 98% or about 99% identical to polynucleotides encoding the fusion proteins of the current invention.

As used herein, “identity” is a measure of the identity of nucleotide sequences or amino acid sequences compared to a reference nucleotide or amino acid sequence, usually a wild-type sequence. In general, the sequences are aligned so that the highest order match is obtained. “Identity” per se has an art-recognized meaning and can be calculated using published techniques. (See, e.g., Computational Molecular Biology, Lesk, A. M., ed., Oxford University Press, New York (1988); Biocomputing: Informatics And Genome Projects, Smith, D. W., ed., Academic Press, New York (1993); Computer Analysis of Sequence Data, Part I, Griffin, A. M., and Griffin, H. G., eds., Humana Press, New Jersey (1994); von Heinje, G., Sequence Analysis In Molecular Biology, Academic Press (1987); and Sequence Analysis Primer, Gribskov, M. and Devereux, J., eds., M Stockton Press, New York (1991)). While there exist several methods to measure identity between two polynucleotide or polypeptide sequences, the term “identity” is well known to skilled artisans (Carillo, H. & Lipton, D., Siam J Applied Math 48:1073 (1988)). Methods commonly employed to determine identity or similarity between two sequences include, but are not limited to, those disclosed in Guide to Huge Computers, Martin J. Bishop, ed., Academic Press, San Diego (1994) and Carillo, H. & Lipton, D., Siam J Applied Math 48:1073 (1988). Computer programs may also contain methods and algorithms that calculate identity and similarity. Examples of computer program methods to determine identity and similarity between two sequences include, but are not limited to, GCS program package (Devereux, J., et al., Nucleic Acids Research 12(i):387 (1984)), BLASTP, BLASTN, FASTA (Atschul, S. F., et al., J Molec Biol 215:403 (1990)).

A polynucleotide having a nucleotide sequence at least, for example, about 95% “identical” to a reference nucleotide sequence encoding a periplasmic binding protein, for example, GGBP, is understood to mean that the nucleotide sequence of the polynucleotide is identical to the reference sequence except that the polynucleotide sequence may include up to about five point mutations per each 100 nucleotides of the reference nucleotide sequence encoding the wild-type GGBP being used as the reference sequence. In other words, to obtain a polynucleotide having a nucleotide sequence at least about 95% identical to a reference nucleotide sequence, up to about 5% of the nucleotides in the reference sequence may be deleted or substituted with another nucleotide, or a number of nucleotides up to about 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

The present invention also relates to vectors that include DNA molecules of the present invention, host cells that are genetically engineered with vectors of the invention and the production of proteins of the invention by recombinant techniques.

Host cells can be genetically engineered to incorporate nucleic acid molecules that are free within the nucleus of the cell (transiently transfected) or incorporated within the chromosome of the cell (stably transfected) and express proteins of the present invention. The polynucleotides may be introduced alone or with other polynucleotides. Such other polynucleotides may be introduced independently, co-introduced or introduced joined to the polynucleotides of the invention.

In accordance with this aspect of the invention, the vector may be, for example, a plasmid vector, a single-or double-stranded phage vector, or a single-or double-stranded RNA or DNA viral vector. Such vectors may be introduced into cells as polynucleotides, preferably DNA, by well-known techniques for introducing DNA and RNA into cells. Viral vectors may be replication competent or replication defective. In the latter, case viral propagation generally will occur only in complementing host cells.

Preferred among vectors, in certain respects, are those for expression of polynucleotides and proteins of the present invention. Generally, such vectors comprise cis-acting control regions effective for expression in a host operatively linked to the polynucleotide to be expressed. Appropriate trans-acting factors either are supplied by the host, supplied by a complementing vector or supplied by the vector itself upon introduction into the host.

A great variety of expression vectors can be used to express the proteins of the invention. Such vectors include chromosomal, episomal and virus-derived vectors, e.g., vectors derived from bacterial plasmids, from bacteriophage, from yeast episomes, from yeast chromosomal elements, from viruses such as adeno-associated virus, lentivirus, baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. All may be used for expression in accordance with this aspect of the present invention. Generally, any vector suitable to maintain, propagate or express polynucleotides or proteins in a host may be used for expression in this regard.

The DNA sequence in the expression vector is operatively linked to appropriate expression control sequence(s) including, for instance, a promoter to direct mRNA transcription. Representatives of such promoters include, but are not limited to, the phage lambda PL promoter, the E. coli lac, trp and tac promoters, HIV promoters, the SV40 early and late promoters and promoters of retroviral LTRs, to name just a few of the well-known promoters. In general, expression constructs will contain sites for transcription, initiation and termination and, in the transcribed region, a ribosome binding site for translation. The coding portion of the mature transcripts expressed by the constructs will include a translation initiating AUG at the beginning and a termination codon (UAA, UGA or UAG) appropriately positioned at the end of the polypeptide to be translated.

In addition, the constructs may contain control regions that regulate, as well as engender expression. Generally, such regions will operate by controlling transcription, such as repressor binding sites and enhancers, among others.

Vectors for propagation and expression generally will include selectable markers. Such markers also may be suitable for amplification or the vectors may contain additional markers for this purpose. In this regard, the expression vectors preferably contain one or more selectable marker genes to provide a phenotypic trait for selection of transformed host cells. Preferred markers include dihydrofolate reductase or neomycin resistance for eukaryotic cell culture, and tetracycline, kanamycin or ampicillin resistance genes for culturing E. coli and other bacteria.

The vector containing the appropriate DNA sequence, as well as an appropriate promoter, and other appropriate control sequences, may be introduced into an appropriate host using a variety of well-known techniques suitable to expression therein of a desired polypeptide. Representative examples of appropriate hosts include bacterial cells, such as E. coli, Streptomyces and Salmonella typhimurium cells; fungal cells, such as yeast cells; insect cells such as Drosophila S2 and Spodoptera Sf9 cells; animal cells such as CHO, COS and Bowes melanoma cells; and plant cells. Hosts for of a great variety of expression constructs are well known, and those of skill in the art will be enabled by the present disclosure to select an appropriate host for expressing one of the proteins of the present invention.

Examples of vectors for use in bacteria include, but are not limited to, pQE70, pQE60 and pQE-9, available from Qiagen (Valencia, Calif.); pBS vectors, Phagescript vectors, Bluescript vectors, pNHSA, pNH16a, pNH18A, pNH46A, available from Stratagene (La Jolla, Calif.); ptrc99a, pKK223-3, pKK233-3, pDR540, pRIT5 available from Amersham-Pharmacia Biotech (Piscataway, N.J.); and pEGFP-C1, pEYFP-C1, pDsRed2-C 1, pDsRed2-Express-C1, and pAcGFP1, pAcGFP-C1, pZsYellow-C1, available from Clontech (Palo Alto, Calif.). Examples of eukaryotic vectors include, but are limited to, pW-LNEO, pSV2CAT, pOG44, pXT1 and pSG available from Stratagene; pSVK3, pBPV, pMSG and pSVL available from Pharmacia; and pCMVDsRed2-express, pIRES2-DsRed2, pDsRed2-Mito, pCMV-EGFP available from Clontech. Many other commercially available and well-known vectors are available to those of skill in the art. Selection of appropriate vectors and promoters for expression in a host cell is a well-known procedure and the requisite techniques for expression vector construction, introduction of the vector into the host and expression in the host are routine skills in the art.

The present invention also relates to host cells containing the above-described constructs. The host cell can be a higher eukaryotic cell, such as a mammalian cell, or a lower eukaryotic cell, such as a yeast cell, or the host cell can be a prokaryotic cell, such as a bacterial cell. The host cell can be stably or transiently transfected with the construct.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods in Molecular Biology (1986).

The proteins of the current invention may be expressed in a modified form and may include not only additional fusions, but also secretion signals and other heterologous functional regions. Thus, for instance, a region of additional amino acids, particularly charged amino acids, may be added to the N-terminus of the protein to improve stability and persistence in the host cell, during purification or during subsequent handling and storage. Also, a region also may be added to the protein to facilitate purification. Such regions may be removed prior to final preparation of the protein. The addition of peptide moieties to proteins to engender secretion or excretion, to improve stability and to facilitate purification, among others, are familiar and routine techniques in the art. A preferred fusion protein comprises a heterologous region from immunoglobulin that is useful to solubilize proteins. For example, EP A0464 533 (Canadian counterpart 2045869) discloses fusion proteins comprising various portions of constant region of immunoglobin molecules together with another human protein or part thereof. In many cases, the Fc part in a fusion protein is thoroughly advantageous for use in therapy and diagnosis and thereby results, for example, in improved pharmacokinetic properties (EP A0232 262). On the other hand, for some uses, it would be desirable to be able to delete the Fc part after the fusion protein has been expressed, detected and purified in the advantageous manner described.

The fusion proteins of the current invention can be recovered and purified from recombinant cell cultures by well-known methods including, but not limited to, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) may also be employed for purification. Well-known techniques for refolding protein may be employed to regenerate active conformation when the fusion protein is denatured during isolation and/or purification.

Fusion proteins of the present invention include, but are not limited to, products of chemical synthetic procedures and products produced by recombinant techniques from a prokaryotic or eukaryotic host, including, for example, bacterial, yeast, higher plant, insect and mammalian cells. Depending upon the host employed in a recombinant production procedure, the fusion proteins of the present invention may be glycosylated or may be non-glycosylated. In addition, fusion proteins of the invention may also include an initial modified methionine residue, in some cases as a result of host-mediated processes.

The fusion proteins may be used in accordance with the present invention for a variety of applications, particularly those useful in detecting or monitoring an analyte. Additional applications relate to diagnosis and to treatment of disorders of cells, tissues and organisms.

The current invention also relates to methods of producing a protein comprising culturing the host cells of the invention under conditions such that said protein is expressed, and recovering said protein. The culture conditions required to express the proteins of the current invention are dependent upon the host cells that are harboring the polynucleotides of the current invention. The culture conditions for each cell type are well-known in the art and can be easily optimized, if necessary.

The present invention also relates to kits useful for monitoring an analyte in a sample. The kits of the current invention comprise at least one composition (fusion protein with a labeling moiety) of the current invention. The kit may also comprise instructions or written material to aid the user.

### EXAMPLE

### Example 1

### Preparation of Mutant GGBP and Fusion Constructs

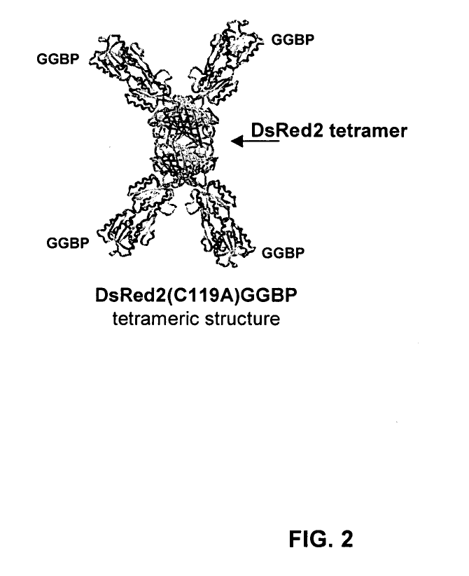

Plasmid pTZ18R contains the MgLB gene from E. coli strain JM109. The GGBP gene was amplified from pTZ18R. The GGBP gene was ligated into the pQE70 plasmid to create a histidine-tagged protein that is wild-type in sequence, except for a lysine-to-arginine change at amino acid position 309, and the addition of a serine at amino acid position 310, before the six histidines at the C-terminus. The DsRed2 gene was amplified from (pDsRed2) and ligated to the N-terminus of the GGBP gene. A short three-alanine linker was engineered into the construct between the fluorescent protein and the histidine-tagged GGBP. Mutations of the GGBP and/or the fluorescent protein were generated in the construct by standard methods. For example, PCR was performed using primers that substitute codon(s) at or near the primary glucose contact sites. This removes the cysteine residue from the DsRed2 portion of the fusion so that when the fusion is fluorophore labeled the label will be site-specifically conjugated to GGBP only. All proteins were histidine-fusions and sequences were confirmed by sequencing. A representation the dsRed2/GGBP fusion protein tetramer is shown in FIG. 2. This was created using coordinates from crystal structures of the two individual proteins (PDB ID's: 1GGX and 2GBP respectively).

### Example 2

### Purification of Fusion Protein Comprising Mutant GGBP and DsRed2

The GGBP was expressed from E. coli strain Sg13009. After E. coli induction for 72 hours, the bacteria were lysed. The lysate was cleared by centrifugation and the DsRed2(C119A)GGBP(E149C,L238C) fusion protein was purified by immobilized metal affinitive chromatography (IMAC) using Talon (cobalt-based) Resin from Clontech. The fusion protein was concentrated using a 100 kDa cutoff filter. The protein was then dialyzed at 4° C. into a solution containing 1M NaCl, 10 mM Tris-HCl, and 50 mM NaPO4 (pH 8) and stored at 20° C.

### Example 3

### Labeling of the Fusion Protein

Fluorophore coupling to a thiol-reactive dye was performed by site-specifically attaching the dye through a covalent bond at a cysteine residue. The fusion was first treated with dithiothreitol, and then a 10-fold molar excess of freshly prepared fluorophore (in this case acrylodan) in dimethyl sulfoxide was subsequently added. The mixture was incubated for four hours and any unreacted dye was removed by size-exclusion column chromatography and/or dialysis. The efficiency of the coupling of the dye to the protein was determined by absorbance.

### Example 4

### Measuring the Fluorescence Intensity and Glucose Affinity for Various Compositions of the Current Invention

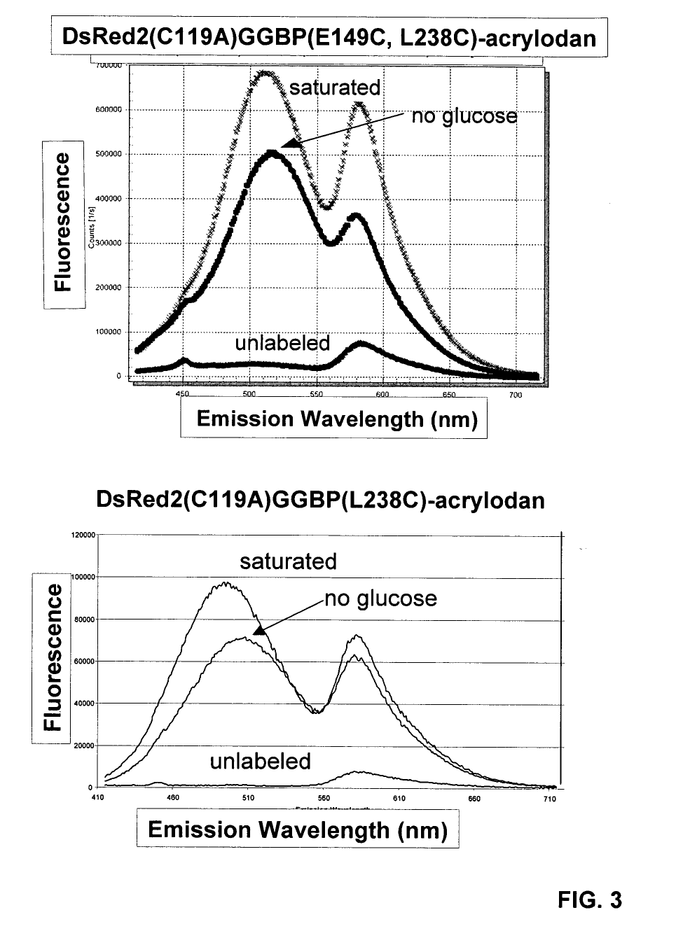

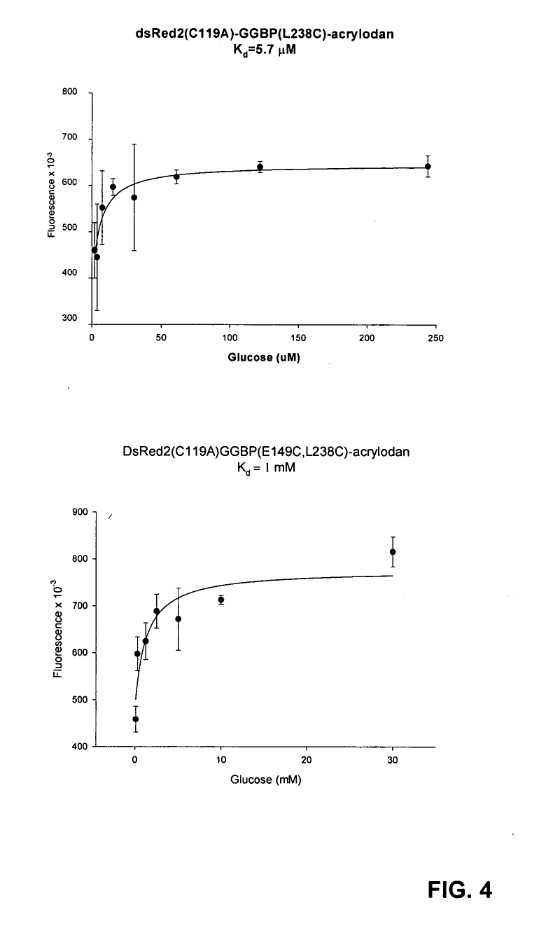

A fluorescence assay was used to determine the affinity of the fusion protein to glucose and to assess the intensity of the fluorescence response. To determine glucose affinities, the acrylodan-labeled fusion protein was incubated with increasing amounts of glucose. For glucose affinity determination of DsRed2(C119A)GGBP (E149C,L238C)-acrylodan, 0.5 μM of the labeled fusion protein was placed in saline solution with or without glucose. Samples were assayed in triplicate and contained either 0, 0.1, 1.0, 2.5, 5.0, 10.0, 20.0, 30.0 or 100.0 mM glucose. Using a spectrofluorometer, the samples were excited at 390 nm and the emission scanned from about 430 to about 700 nm. The acrylodan and DsRed2 emissions were read at 520 nm and 583 nm, respectively. A non-dye-labeled negative control of the fusion protein was tested to confirm fluorescent resonance energy transfer (FIG. 3). To determine the affinity of the fusion for the analyte (in this example glucose), the emission of DsRed2 was graphed (FIG. 4) according the equation:
f=F∞+Fₒₜ/(1+(x/K_{d}))
where K_{d} is equal to the glucose concentration at the half-maximal response. For DsRed2(C119A)GGBP(E149C,L238C)-acrylodan, a glucose affinity of about 1 mM was demonstrated.

Other DsRed2(C119A)-GGBP fusions were constructed with amino acid substitutions at other GGBP sites and then purified and labeled with acrylodan by methods similar to the preceding examples. Titration of the acrylodan labeled proteins vs. saturating concentrations of glucose gave the following data. The performance of the proteins for ratiometric measurements was determined according to the formula:
QR=[[(I_{ac}/I_{R})−(Io_{ac}/Io_{R})]/(Io_{ac}/Io_{R})]* 100

In this formula, QR=Ratiometric Quotient (%); I_{ac}=acrylodan fluorescence emission intensity (+glucose); I_{R}=DsRed2 fluorescence emission intensity (+glucose); Io_{ac}=Acrylodan fluorescence emission intensity (no glucose); Io_{R}=DsRed2 fluorescence emission intensity (no glucose). The raw data and calculation of QR are given in Table II. Generally speaking, the higher the absolute value of QR, the greater the ratiometric change is that accompanies ligand binding (the ligand being glucose in these particular examples).

TABLE II
Performance of Variants of DsRed2 (C119A) - GGBP-acrylodan

| Fusion | | | | | | |
|---|---|---|---|---|---|---|
| Protein | Io_{ac} | I_{ac} | Io_{R} | I_{R} | QR | Kd |
| N43C | 30330 | 34,500 | 15000 | 17100 | −0.2% | ND* |
| E149C | 27500 | 33000 | 55900 | 62200 | +7.8% | 0.04 uM |
| E149C/ | 14600 | 18300 | 12000 | 16000 | −6.0% | 1 mM |
| L238C | | | | | | |
| A213C | 63750 | 60500 | 40800 | 39200 | −1.2% | ND |
| M216C | 45250 | 44750 | 22250 | 23125 | −4.8% | ND |
| L238C | 69400 | 96950 | 63000 | 72200 | +21.9% | 5.7 uM |

*ND = Data not determined

Acrylodan emission was typically measured at 495 nm and DsRed emission at 582 nm. Data was not corrected for fluorescence of DsRed2 in the absence of acrylodan upon 390 nm excitation (typically only 10-15% of Io_{R} values). All proteins were labeled with approximately one dye per GGBP except the third example, the E149C/L238C mutant, was labeled with approximately 2 dyes per GGBP.

### Example 5

### Measuring the Concentration of Glucose in a Sample Using the Compositions and Methods of the Current Invention

To measure unknown amounts of glucose in samples, the fusion protein was added to the sample and excited at the excitation wavelength (390 nm for acrylodan). Then the fluorescence emission was recorded. To quantify the analyte, the fluorescence of the sample was compared to known fluorescent responses from standard glucose solutions. The unknown glucose concentration in the sample was determined from formulas generated describing the standard curve. Additionally, the PBP could be expressed in the sample or cell prior to fluorescence analysis.

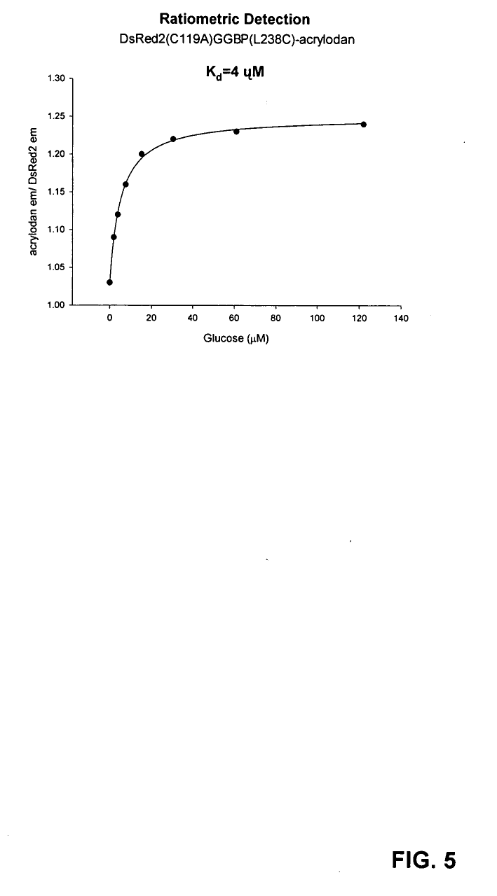

For DsRed2(C119A)-GGBP(L238C)-acrylodan, a standard curve was generated for glucose solutions from 0-8 μM. For unknown determinations, the simplest calculations use a linear regression equation (y=mx+b). Linear regression was performed on the linear portion of the binding curves for both the absolute fluorescence data (FIG. 4) and data from the ratio of the acrylodan emission and the DsRed2 emission (Table III and FIG. 5). To determine the glucose concentration for unknowns, the DsRed2(C119A)-GGBP(L238C)-acrylodan was added to the sample and the fluorescence reading was taken. Table IV lists results for glucose determinations in samples.

TABLE III
DsRed2(C119A)-GGBP(L238C)-Acrylodan Standard Curve

| | | |
|---|---|---|
| DsRed2 582 nm | y = 7513.8x + 654023 | R² = 0.8879 |
| Ratio (495 nm/582 nm) | y = 0.0115x + 1.1218 | R² = 0.9705 |

TABLE IV
Determination Of Glucose Concentration of Unknown Samples

| Unknown [glucose] | DsRed2 582 nm | Ratio (495 nm/582 nm) |
|---|---|---|
| Actual (μM) | Determined | Determined |
| 4 | 4 | 5 |
| 8 | 9 | 9 |
| 16 | 16 | 16 |

### Example 6

### Example of Fusion Protein Reversibility and Continuous Monitoring of an Analyte

The ability to continuously monitor a sample during analyte concentration fluctuations in the sample environment over time is a unique characteristic of PBPs. Continuous monitoring by PBPs is possible due to the reversible ligand-binding capabilities of the receptors. To demonstrate how glucose can be monitored continuously in a single sample, DsRed2(C119A)-GGBP(L238C) was placed in a solution that was absent of glucose and the fluorescence ratio was determined. Glucose was then added to a concentration of 64 μM and a fluorescence reading was recorded. The sample was then placed in a dialysis chamber and dialyzed to remove glucose. After dialysis, the sample was again tested for fluorescence emission. This demonstrated that the emission ratio had returned to near the initial reading taken in the absence of glucose. Finally, the glucose concentration was increased to 71 μM and the emission ratio also increased, indicating the presence of glucose (Table V).

TABLE V
Continuous Monitoring of Glucose

| Time | Glucose | Ratio |
|---|---|---|
| (hr) | (μM) | (495 nm/582 nm) |
| 0 | 0 | 0.95 |
| 0.25 | 64 | 1.33 |
| 3.25 | 0 | 0.89 |
| 3.5 | 71 | 1.27 |

1. A method for quantifying an analyte in a sample, said method comprising:
a) administering a fusion protein to said sample, said fusion protein comprising a functional periplasmic binding protein (PBP), at least one labeling moiety and at least one fluorescent protein; and
b) measuring the luminescence of said fluorescent fusion protein, wherein said luminescence is indicative of the amount of analyte in said sample.

2. The method of claim 1, wherein said measuring is performed at more than one time point in the same sample.

3. The method of claim 1, wherein said measurement can be made continuously.

4. The method of claim 1, wherein said fusion protein binds reversibly to said analyte.

5. The method of claim 1, wherein said sample is a biological fluid.

6. The method of claim 1, wherein said quantifying comprises calculating a ratio of the fluorescence of said at least one fluorescent protein to the fluorescence of said at least one labeling moiety.

7. The method of claim 1, wherein said fusion protein comprises at least two fluorescent proteins.

8. The method of claim 7, wherein said quantifying comprises calculating a ratio of the fluorescence of said at least two fluorescent proteins.

9. The method of claim of claim 8, wherein said at least two fluorescent proteins are not identical.

10. The method of claim 1, wherein said functional PBP is selected from the group consisting of glucose-galactose binding protein (GGBP), maltose binding protein (MBP), ribose binding protein (RBP), arabinose binding protein (ABP), dipeptide binding protein (DPBP), glutamate binding protein (GluBP), iron binding protein (FeBP), histidine binding protein (HBP), phosphate binding protein (PhosBP), glutamine binding protein (GBP), oligopeptide binding protein (OppA) and derivatives thereof.

11. The method of claim 10, wherein said functional PBP is GGBP or a derivative thereof.

12. The method of claim 10, wherein said analyte is glucose.

13. The method of claim 1, wherein said at least one fluorescent protein is selected from the group consisting of a green fluorescent protein (GFP), red-shifted GFP (rs-GFP), a red fluorescent protein (RFP), a yellow fluorescent protein (YFP), a cyan fluorescent protein (CFP), a blue fluorescent protein (BFP), enhanced versions thereof and mutations thereof.

14. The method of claim 13, wherein said at least one fluorescent protein is RFP.

15. The method of claim 14, wherein said RFP is selected from the group consisting of DsRed2, HcRed1, DsRed-Express and mutations thereof.

16. The method of claim 15, wherein said RFP is the DsRed2 mutant DsRed2(C119A).

17. The method of claim 1, wherein said labeling moiety is a fluorophore.

18. The method of claim 17, wherein said fluorophore is selected from the group consisting of fluorescein, acryoldan, rhodamine, BODIPY, eosin, pyrene, acridine orange, PyMPO, alexa fluor 488, alexa fluor 532, alexa fluor 546, alexa fluor 568, alexa fluor 594, alexa fluor 555, alexa fluor 633, alexa fluor 647, alexa fluor 660 and alexa fluor 680.

19. A composition comprising a fusion protein portion and at least one labeling moiety, said fusion protein portion comprising a functional periplasmic binding protein and at least one fluorescent protein.

20. The composition of claim 19, wherein said functional periplasmic binding protein is selected from the group consisting of glucose-galactose binding protein (GGBP), maltose binding protein (MBP), ribose binding protein (RBP), arabinose binding protein (ABP), dipeptide binding protein (DPBP), glutamate binding protein (GluBP), iron binding protein (FeBP), histidine binding protein (HBP), phosphate binding protein (PhosBP), glutamine binding protein, oligopeptide binding protein (OppA) and derivatives thereof.

21. The composition of claim 20, wherein said functional PBP is GGBP or a derivative thereof.

22. The composition of claim 21, further comprising at least one additional fluorescent protein.

23. The composition of claim 22, wherein said at least two fluorescent proteins are not identical.

24. The composition of claim 19, wherein said at least one fluorescent protein is selected from the group consisting of a green fluorescent protein (GFP), a red-shifted GFP (rs-GFP), a red fluorescent protein (RFP), a yellow fluorescent protein (YFP), a cyan fluorescent protein (CFP), a blue fluorescent protein (BFP), enhanced versions thereof, and mutations thereof.

25. The composition of claim 24, wherein said at least one fluorescent protein is RFP.

26. The composition of claim 25, wherein said RFP is selected from the group consisting of DsRed2, HcRed1, dsRed-Express and mutations thereof.

27. The composition of claim 26, wherein said RFP is the DsRed2 mutant DsRed2(C119A).

28. The composition of claim 27, wherein said labeling moiety is a fluorophore.

29. The composition of claim 28, wherein said fluorophore is selected from the group consisting of fluorescein, acryoldan, rhodamine, BODIPY, acridine orange, eosin, pyrene, acridine orange, PyMPO, alexa fluor 488, alexa fluor 532, alexa fluor 546, alexa fluor 568, alexa fluor 594, alexa fluor 555, alexa fluor 633, alexa fluor 647, alexa fluor 660 and alexa fluor 680.

30. A vector comprising a nucleic acid sequence coding for the fusion protein portion of the composition of claim 19.

31. A host cell comprising the vector of claim 30.

32. A method of producing a protein, comprising culturing the host cell of claim 31 under conditions such that said protein is expressed, and recovering said protein.

33. A kit for detecting the concentration of an analyte in a sample, said kit comprising the composition of claim 19.

34. The kit of claim 33, wherein said functional periplasmic binding protein is selected from the group consisting of glucose-galactose binding protein (GGBP), maltose binding protein (MBP), ribose binding protein (RBP), arabinose binding protein (ABP), dipeptide binding protein (DPBP), glutamate binding protein (GluBP), iron binding protein (FeBP), histidine binding protein (HBP), phosphate binding protein (PhosBP), glutamine binding protein, oligopeptide binding protein (OppA) and derivatives thereof.

35. The kit of claim 34, wherein said functional periplasmic binding protein is glucose-galactose binding protein (GGBP) or a derivative thereof.

36. The kit of claim 35, wherein said analyte is glucose.

37. The kit of claim 36, wherein said at least one fluorescent protein is selected from the group consisting of a green fluorescent protein (GFP), a red-shifted GFP (rs-GFP), a red fluorescent protein (RFP), a yellow fluorescent protein (YFP), a cyan fluorescent protein (CFP), a blue fluorescent protein (BFP) and enhanced versions thereof.

38. The kit of claim 37, wherein said at least one fluorescent protein is RFP.

39. The kit of claim 38, wherein said RFP is discosoma red fluorescent protein (DsRed2).
